(19) United States
(12) Patent Application Publication
(10) Pub. No.: US 2016/0296558 A1
(43) Pub. Date: Oct. 13, 2016

(54) ADSORBENT FOR REDUCING UREMIC TOXINS IN VIVO

(71) Applicant: BIO-MEDICAL CARBON TECHNOLOGY CO., LTD., Taichung City (TW)

(72) Inventors: Tse-Hao KO, TAIPEI CITY (TW); Jui-Hsiang LIN, TAICHUNG CITY (TW); Wei-Shan HSU, TAICHUNG CITY (TW); Wan-Yu CHUNG, TAICHUNG CITY (TW)

(21) Appl. No.: 14/683,501

(22) Filed: Apr. 10, 2015

(51) Int. Cl. A61K 33/44; B01J 20/28; B01J 20/20; A61K 9/00; A61K 45/06

(52) U.S. Cl. CPC A61K 33/44; A61K 9/0053; A61K 45/06; B01J 20/205; B01J 20/28023; B01J 20/28061; B01J 20/28064; B01J 20/28066

(57) ABSTRACT

An adsorbent for reducing uremic toxins in vivo includes polyacrylonitrile-based activated carbon fibers having the following properties: a) an average diameter of 5 μm to 30 μm; b) a BET specific surface area of more than 390 m²/g; c) a total acidic group content of larger than 1.2 meq/g or a total basic group content of larger than 1 meq/g. Because the adsorbent of the present disclosure has a higher adsorption capacity for precursors of uremic toxins than for the normal enzyme protein in intestinal tract, the adsorbent of the present disclosure can effectively prevent the deterioration of renal function, and thus can be used as a therapeutic agent and a preventive for kidney disease.

### BACKGROUND OF THE INVENTION

1. Field of the Invention

The present disclosure relates generally to an orally administered substance for treating or preventing kidney disease and more particularly, to an adsorbent capable of reducing uremic toxins in vivo, which is used to prevent the deterioration of renal function or to reduce the deterioration rate of renal function.

2. Description of the Related Art

The metabolic wastes excreted from kidney are known as uremic toxins. The concentration of the uremic toxin of the serum is an index of renal function. One of the uremic toxins, indoxyl sulfate, is regarded as one of the major reasons for the deterioration of chronic kidney disease. Generally speaking, tryptophan obtained from food is metabolized into indole by intestinal bacteria, and then the indole is metabolized into indoxyl sulfate in the liver, which in turn is excreted from kidney. When the renal function deteriorates, indoxyl sulfate and other uremic toxins, such as creatinine, blood urea nitrogen, etc., accumulates in the body, leading to kidney failure eventually. For a kidney-failure patient, kidney transplantation or dialysis treatment is necessary to sustain the patient's life. However, kidney transplantation is not always easy to carry out, and dialysis treatment tends to cause a complication such as thrombosis. Therefore, if the amount of uremic toxins accumulated in the body at the initial stage of deterioration of renal function can be minimized, the deterioration rate of renal function can be reduced, thereby avoiding the need of dialysis treatment.

When fed into intestine, an excellent adsorbent for reducing uremic toxins preferably has a high adsorption capacity for precursors of uremic toxins such as indole, but a low adsorption capacity for enzyme protein such as lipase presented in intestinal tract. An adsorbent having such selectivity can thus reduce the amount of uremic toxins accumulated in the body and can maintain the normal function of gastro-intestinal tract. A conventional adsorbent has a poor adsorption capacity for precursors of uremic toxins and has no selectivity, thus it can neither effectively reduce the deterioration of renal function nor maintain the normal function of gastro-intestinal tract.

### SUMMARY OF THE INVENTION

It is an objective of the present disclosure to provide an adsorbent for reducing uremic toxins in vivo, which is capable of effectively removing precursors of uremic toxins in gastro-intestinal tract under the condition that the enzyme protein in intestinal tract may not be affected, reducing the rate of accumulation of uremic toxins in vivo, reducing the deterioration rate of renal function, and avoiding the need of dialysis treatment.

To attain the above-mentioned objective, the present disclosure provides an adsorbent, which comprises polyacrylonitrile-based activated carbon fibers having the following properties: a) an average diameter of 5-30 μm; b) a BET specific surface area of more than 390 m²/g; and c) a total acidic group content of larger than 1.2 meq/g or a total basic group content of larger than 1 meq/g.

In comparison with the conventional adsorbent, the adsorbent of the present disclosure has a higher adsorption capacity for precursors of uremic toxins than for enzyme protein in intestinal tract; it can effectively reduce the amount of uremic toxins accumulated in the body while maintaining the normal function of the gastro-intestinal tract; it can be used as a therapeutic agent for kidney disease to reduce the deterioration rate of renal function or can be used as a preventive for kidney disease to avoid the need of dialysis treatment.

### BRIEF DESCRIPTION OF THE DRAWINGS

FIG. 1 is a scanning electron microscopic (SEM) image of polyacrylonitrile-based activated carbon fibers used in an example 1 of the present disclosure.

FIG. 2 is a diagram showing the adsorption percentages of indole and lipase in vitro of examples 1 to 5 of the present disclosure and a comparative example.

FIG. 3 is a diagram of an in vivo test showing the concentration changes of indoxyl sulfate in serums of a rat in a normal group (WT), a rat in a control group (Nep), and a rat in an experimental group (Nep-ACF).

FIG. 4 is a diagram of an in vivo test showing the concentration changes of blood urea nitrogen in serums of the rat in the normal group (WT), the rat in the control group (Nep), and the rat in the experimental group (Nep-ACF).

FIG. 5 is a diagram of an in vivo test showing the concentration changes of creatinine in serums of the rat in the normal group (WT), the rat in the control group (Nep), and the rat in the experimental group (Nep-ACF).

FIG. 6 is a stained slice image of a kidney of the rat in the normal group.

FIG. 7 is a stained slice image of a kidney of the rat in the control group.

FIG. 8 is a stained slice image of a kidney of the rat in the experimental group that is fed with the adsorbent according to example 1 of the present disclosure.

### DETAILED DESCRIPTION OF EMBODIMENTS

The technical features and the effect of the present disclosure will become more fully understood from the detailed description and embodiments given herein below and the properties mentioned in each embodiment are determined by the following methods.

Average diameter and average length were determined by a scanning electron microscope (S-4800, from Hitachi company, Japan).

BET specific surface area and percentages of micropores (diameter of less than 2 nm), mesopores (diameter of between 2 nm and 50 nm), and macropores (diameter of larger than 50 nm) were determined by a high-resolution specific surface area analyzer (ASAP2020, from Micromeritics company, America).

Density was determined by a true density measurement method including the steps of vacuumizing a closed space in which an adsorbent is placed, removing the water vapor filled in the pores of the adsorbent and then refilling nitrogen gas, and calculating the amount of the refilled nitrogen gas to determine the true volume and weight of the adsorbent to obtain the true density.

Determination of total acidic group content was carried out as follows. 1 g of adsorbent was added into 50 mL of 0.05N aqueous sodium hydroxide (NaOH), the mixture was shaken for 48 hours at room temperature. After filtration, the filtrate was titrated to neutral with 0.05N aqueous hydrogen chloride (HCl). The total acidic group content (per gram of sample, meq/g) can be calculated from the amount of HCl used for titration. Determination of total basic group content was carried out as follows. 1 g of adsorbent was added into 50 mL of 0.05N aqueous HCl, the mixture was shaken for 24 hours at room temperature. After filtration, the filtrate was titrated to neutral with 0.05N aqueous NaOH. The total basic group content (per gram of sample, meq/g) can be calculated from the amount of NaOH used for titration.

Adsorption test of indole and lipase in vitro was carried out in a simulated solution simulating intestinal tract condition. 0.01 g of adsorbent was added into 10 mL of the simulated solution containing 100 ppm indole, 100 ppm lipase, and 5 wt % bile acid. After reacting for three hours at 37° C., the residual concentration of the indole and lipase in the simulated solution was measured. The adsorption percentage was calculated based on the following equation:

$$adsorption percentage ( \% ) = 100 - ( \frac{residual concentration}{original concentration} ) \times 100$$

Regarding evaluation of food safety, BALB/c mice were fed with 5 wt % of the adsorbent of the present disclosure relative to the total weight of the feed each day for 30 days, and then the survival rate, behavior and color of feces of the mice were monitored to evaluate the food safety.

Adsorption test of precursors of uremic toxins in vivo was carried out as follows. Three male Sprague-Dawley rats, weighing between 200 g and 250 g, were divided into a normal group (WT), a control group (Nep) and an experimental group (Nep-ACF). The rat in the normal group was not subjected to kidney excision and was fed with normal feed; the rat in the control group was subjected to 5/6 kidney excision and was fed with normal feed; and the rat in the experimental group was subjected to 5/6 kidney excision and was fed with the feed containing 5 wt % of the adsorbent of the present disclosure. These rats were fed for 10 weeks and the concentrations of indoxyl sulfate, blood urea nitrogen, and creatinine in serums were measured every week.

After the adsorption test of precursors of uremic toxins in vivo was completed, pathological diagnosis of kidney tissue was conducted by obtaining the kidney tissue slices of these rats, which were stained by hematoxylin and eosin stain (H&E stain). The severity grading scheme of pathological diagnosis of kidney tissue was based on the method recited in Toxicologic Pathology, vol. 30, no. 1, pp 93-96, 2002, Shackelford et al. In the aforesaid method, a specific range of the kidney tissue was determined and graded, by the extent of the renal tubular degeneration or regeneration, into Grade 1 (minimal, less than 1%); Grade 2 (slight, 1 to 25%); Grade 3 (moderate, 26 to 50%); Grade 4 (moderately severe, 51-75%); and Grade 5 (severe, 76 to 100%).

### Example 1

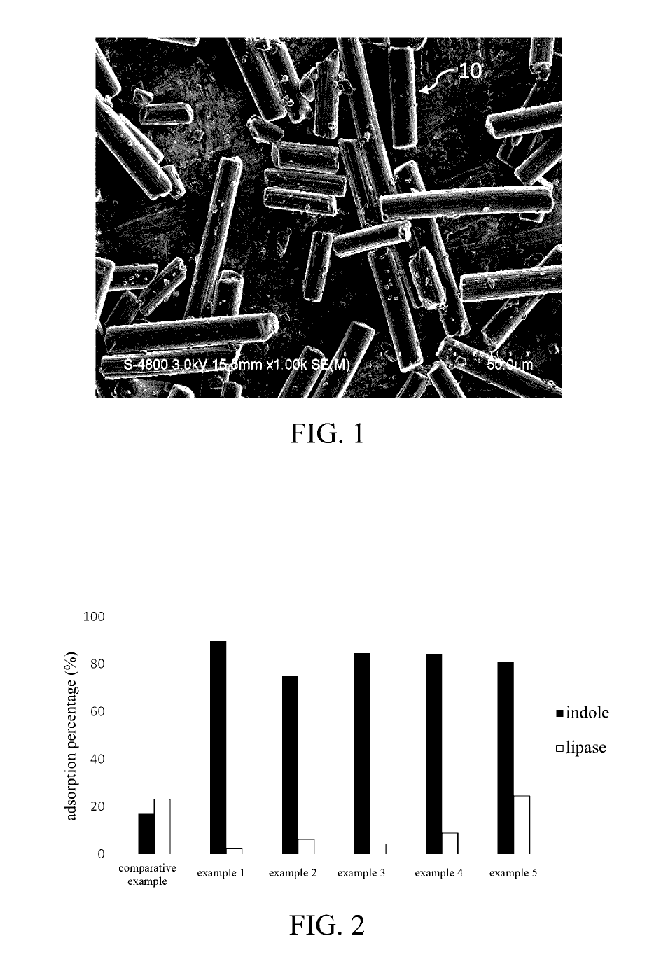

An adsorbent for reducing uremic toxins according to example 1 is a capsule in which polyacrylonitrile-based activated carbon fibers are encapsulated. The polyacrylonitrile-based activated carbon fibers were prepared by treating an oxidized polyacrylonitrile-based carbon fiber material with carbon dioxide gas containing water vapor at a temperature of 1000° C. for 5 minutes and grinding the carbon fiber material thus treated. In this example, the oxidized polyacrylonitrile-based carbon fiber material is formed by oxidizing a polyacrylonitrile-based carbon fiber cloth (Panex® 30, from Zoltek Companies, Inc.) which contains 90 wt % of polyacrylonitrile and 10 wt % of Rayon or petroleum pitch. The polyacrylonitrile-based activated carbon fibers thus prepared have an average diameter of 7.6 μm; a BET specific surface area of 964 m²/g; a density of 2.13 g/m³; a percentage of micropores of 22%, a percentage of mesopores of 78%, and a percentage of macropores of 0%; an average length of 23.2±6.9 μm; a total acidic group of 1.092 meq/g; and a total basic group of 1.30 meq/g. FIG. 1 is the SEM image showing the structure of the polyacrylonitrile-based activated carbon fibers used in example 1.

FIG. 2 is the diagram showing the test result of the adsorption in vitro. As shown in FIG. 2, the adsorbent of example 1 has an adsorption percentage of indole of 89.6% and an adsorption percentage of lipase of 2.33%. Based on the result that the survival rate and behavior of the mice are normal and the colors of feces of the mice are dark, it is deduced that the adsorbent is excreted from gastro-intestinal tract of mice after digestion, and thus the adsorbent has good food safety.

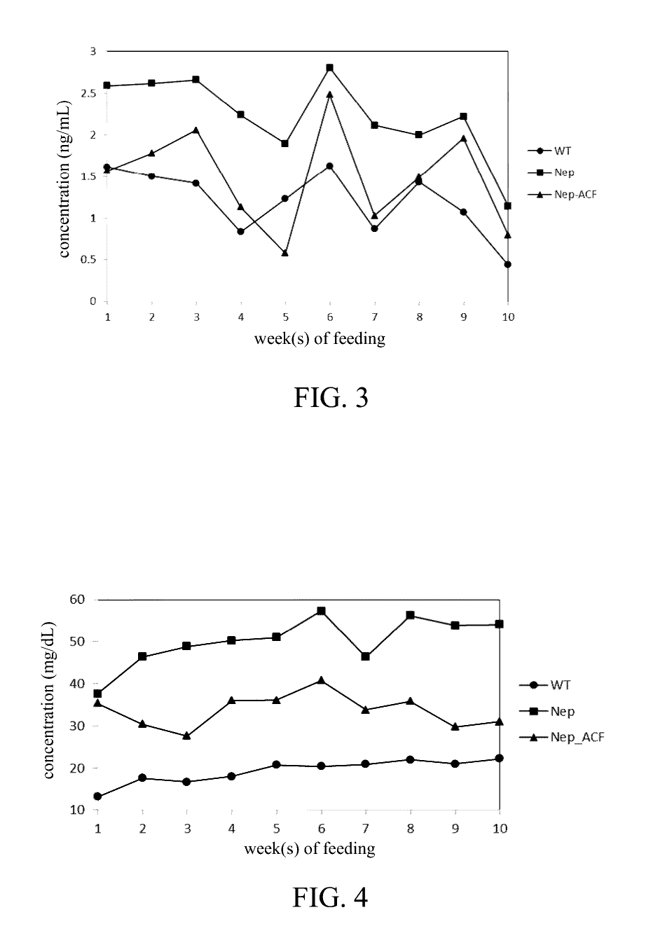

FIG. 3 is the diagram showing the test result of the adsorption of indoxyl sulfate in vivo. As shown in FIG. 3, the concentration of indoxyl sulfate in serum of the rat in Nep-ACF (1.55 ng/mL) is lower than that of the rat in Nep (2.6 ng/mL) after a week. During ten weeks, all of the test results show that the concentrations of indoxyl sulfate in serum of the rat in Nep-ACF are lower than those of the rat in Nep. Therefore, it is proved that the adsorbent of the present disclosure can effectively reduce the amount of uremic toxins accumulated in the body of rat with deteriorated renal function.

FIG. 4 is the diagram showing the test result of the adsorption of blood urea nitrogen in vivo. As shown in FIG. 4, the concentration of blood urea nitrogen in serum of the rat in Nep-ACF (36 mg/dL) is lower than that of the rat in Nep (38 mg/dL) after a week. After ten weeks, the concentration of blood urea nitrogen in serum of the rat in Nep-ACF (30 mg/dL) is slightly higher than that of the rat in WT (22 mg/dL) of about 8 mg/dL, whereas the concentration of blood urea nitrogen in serum of the rat in Nep (56 mg/dL) is higher than that of the rat in WT of about 34 mg/dL. That is, the accumulated concentration of blood urea nitrogen in serum of the rat in Nep-ACF is only 24% of that of the rat in Nep. Therefore, it is proved that the adsorbent of the present disclosure can effectively reduce the amount of blood urea nitrogen accumulated in the body of rat with deteriorated renal function.

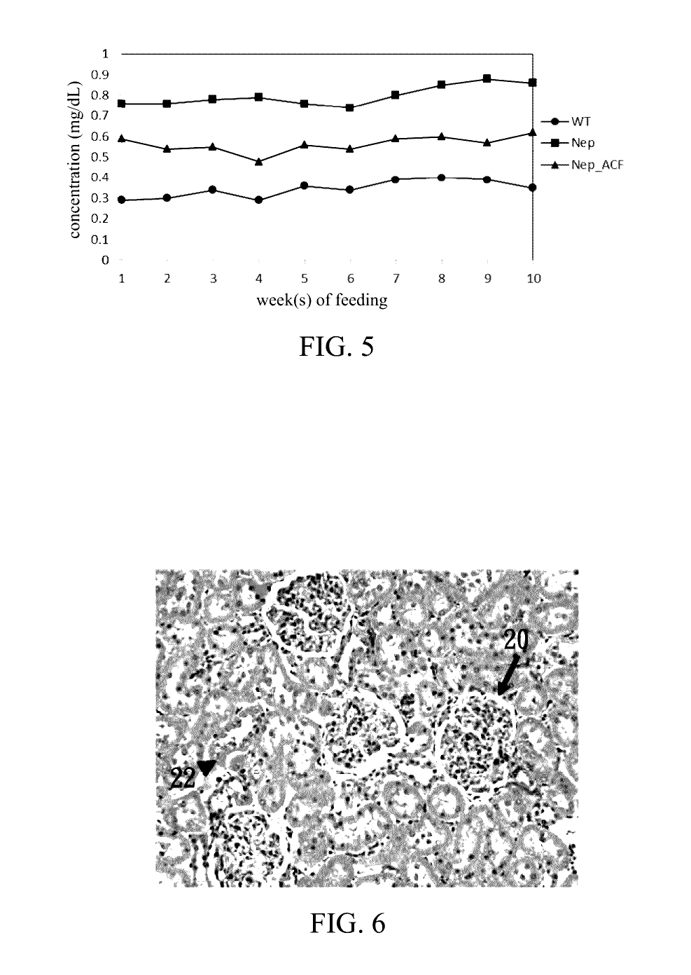

FIG. 5 is the diagram showing the test result of the adsorption of creatinine in vivo. As shown in FIG. 5, the concentration of creatinine in serum of the rat in Nep-ACF (0.6 mg/dL) is lower than that of the rat in Nep (0.75 mg/dL) after a week. After ten weeks, the concentration of creatinine in serum of the rat in Nep-ACF (0.61 mg/dL) is slightly higher than that of the rat in WT (0.36 mg/dL) of about 0.25 mg/dL, whereas the concentration of creatinine in serum of the rat in Nep (0.86 mg/dL) is higher than that of the rat in WT of about 0.5 mg/dL. That is, the accumulated concentration of creatinine in serum of the rat in Nep-ACF is only 50% of that of the rat in Nep. Therefore, it is proved that the adsorbent of the present disclosure can effectively reduce the amount of creatinine accumulated in the body of rat with deteriorated renal function.

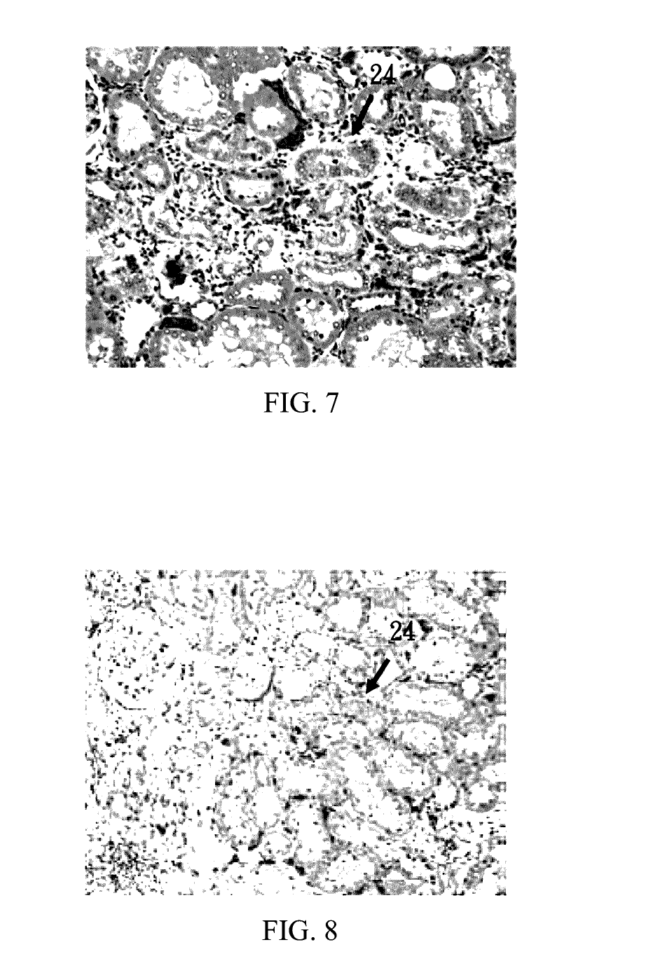

FIGS. 6-8 show the severity of kidney tissue damage of the rats. As shown in FIG. 6, the rat in WT has normal glomerulus 20 and renal tubular 22. FIG. 7 shows that the rat in Nep has degenerated or regenerated renal tubular 24 and has the severity of Grade 3. FIG. 8 shows that the rat in Nep-ACF has degenerated or regenerated renal tubular 24 and has the severity of Grade 2. In comparison with FIGS. 6-8, it is proved that the severity of kidney damage of the rat with deteriorated renal function can be decreased after the rat is fed with the adsorbent of the present disclosure for ten weeks.

In conclusion, the adsorbent of example 1 has a higher adsorption capacity for precursors of uremic toxins than for enzyme protein in intestinal tract, has a good food safety, can effectively reduce the concentration of the uremic toxins in vivo, and can reduce the severity of kidney damage. Accordingly, the adsorbent of example 1 can decrease the deterioration rate of renal function while maintaining the normal function of gastro-intestinal tract to avoid the need of dialysis treatment.

### Comparative Example

A commercially available food-grade activated bamboo charcoal powder is used as an adsorbent of the comparative example. The activated bamboo charcoal powder has an irregular shape; an average particle diameter of 2.9±1.4 μm; a BET specific surface area of 329 m²/g; and a percentage of micropores of 15.7%, a percentage of mesopores of 83.5%, and a percentage of macropores of 0.7%. As shown in FIG. 2, the adsorbent of the comparative example has an adsorption percentage of indole of 17.1% and an adsorption percentage of lipase of 23.25%, that is the adsorbent of the comparative example has a higher adsorption capacity for enzyme protein in intestinal tract than for precursors of uremic toxins. Accordingly, the adsorbent of the comparative example cannot effectively reduce the amount of uremic toxins accumulated in the body and may disturb the normal function of gastro-intestinal tract.

Because the adsorbent of example 1 has a higher adsorption capacity for precursors of uremic toxins and a lower adsorption capacity for enzyme protein in intestinal tract than the adsorbent of the comparative example, the adsorbent of example 1 can reduce the deterioration rate of renal function while maintaining the normal function of the gastro-intestinal tract. In addition to example 1, the following examples 2 to 5 have similar effect.

### Example 2

An adsorbent according to example 2 is a capsule in which polyacrylonitrile-based activated carbon fibers are encapsulated. The polyacrylonitrile-based activated carbon fibers were prepared by oxidizing the polyacrylonitrile-based carbon fiber cloth (Panex® 30, from Zoltek Companies, Inc.); treating the oxidized polyacrylonitrile-based carbon fiber cloth with the carbon dioxide gas containing water vapor at a temperature of 900° C. for 20 minutes; and grinding the carbon fiber thus treated. The polyacrylonitrile-based activated carbon fibers have an average diameter of 9.3 μm; a BET specific surface area of 398 m²/g; a density of 1.749 g/m³; a percentage of micropores of 18%, a percentage of mesopores of 82%, and a percentage of macropores of 0%; an average length of 27.1±2.4 μm; a total acidic group of 1.559 meq/g; and a total basic group of 0.9 meq/g. The test result of the adsorption in vitro is shown in FIG. 2, in which the adsorbent of example 2 has an adsorption percentage of indole of 75.3% and an adsorption percentage of lipase of 6.3%. It can be seen, from the aforesaid result, that the adsorbent of example 2 has a higher adsorption capacity for precursors of uremic toxins than for enzyme protein in intestinal tract.

### Example 3

An adsorbent according to example 3 is a capsule in which polyacrylonitrile-based activated carbon fibers are encapsulated. The polyacrylonitrile-based activated carbon fibers were prepared by oxidizing the polyacrylonitrile-based carbon fiber cloth (Panex® 30, from Zoltek Companies, Inc.); treating the oxidized polyacrylonitrile-based carbon fiber cloth with the carbon dioxide gas containing water vapor at a temperature of 900° C. for 40 minutes; and grinding the carbon fiber thus treated. The polyacrylonitrile-based activated carbon fibers have an average diameter of 8.6 μm; a BET specific surface area of 921 m²/g; a density of 2.043 g/m³; a percentage of micropores of 21%, a percentage of mesopores of 79%, and a percentage of macropores of 0%; an average length of 21.9±1.4 μm; a total acidic group of 1.384 meq/g; and a total basic group of 1.26 meq/g. The test result of the adsorption in vitro is shown in FIG. 2, in which the adsorbent of example 3 has an adsorption percentage of indole of 84.7% and an adsorption percentage of lipase of 4.4%. It can be seen, from the aforesaid result, that the adsorbent of example 3 has a higher adsorption capacity for precursors of uremic toxins than for enzyme protein in intestinal tract.

### Example 4

An adsorbent according to example 4 is a capsule in which polyacrylonitrile-based activated carbon fibers are encapsulated. The polyacrylonitrile-based activated carbon fibers were prepared by oxidizing the polyacrylonitrile-based carbon fiber cloth (Panex® 30, from Zoltek Companies, Inc.); treating the oxidized polyacrylonitrile-based carbon fiber cloth with the carbon dioxide gas containing water vapor at a temperature of 1000° C. for 20 minutes; and grinding the carbon fiber thus treated. The polyacrylonitrile-based activated carbon fibers have an average diameter of 6 μm; a BET specific surface area of 1244 m²/g; a density of 2.153 g/m³; a percentage of micropores of 18%, a percentage of mesopores of 82%, and a percentage of macropores of 0%; an average length of 26.2±2.5 μm; a total acidic group of 1.253 meq/g; and a total basic group of 1.685 meq/g. The test result of the adsorption in vitro is shown in FIG. 2, in which the adsorbent of example 4 has an adsorption percentage of indole of 84.3% and an adsorption percentage of lipase of 8.9%. It can be seen, from the aforesaid result, that the adsorbent of example 4 has a higher adsorption capacity for precursors of uremic toxins than for enzyme protein in intestinal tract.

### Example 5

An adsorbent according to example 5 is a capsule in which polyacrylonitrile-based activated carbon fibers are encapsulated. The polyacrylonitrile-based activated carbon fibers were prepared by oxidizing the polyacrylonitrile-based carbon fiber cloth (Panex® 30, from Zoltek Companies, Inc.); treating the oxidized polyacrylonitrile-based carbon fiber cloth with the carbon dioxide gas containing water vapor at a temperature of 1000° C. for 40 minutes; and grinding the carbon fiber thus treated. The polyacrylonitrile-based activated carbon fibers have an average diameter of 5.6 μm; a BET specific surface area of 1494 m²/g; a density of 2.163 g/m³; a percentage of micropores of 19%, a percentage of mesopores of 81%, and a percentage of macropores of 0%; an average length of 22.7±4.5 μm; a total acidic group of 1.7 meq/g; and a total basic group of 0.969 meq/g. The test result of the adsorption in vitro is shown in FIG. 2, in which the adsorbent of example 5 has an adsorption percentage of indole of 81.2% and an adsorption percentage of lipase of 24.5%. It can be seen, from the aforesaid result, that the adsorbent of example 5 has a higher adsorption capacity for precursors of uremic toxins than for enzyme protein in intestinal tract.

As described above, the adsorbent which comprises the polyacrylonitrile-based activated carbon fibers having an average diameter of 5-30 μm, a BET specific surface area of more than 390 m²/g, a total acidic group content of larger than 1.2 meq/g or a total basic group content of larger than 1 meq/g, can have a higher adsorption capacity for precursors of uremic toxins than for enzyme protein in intestinal tract. As such, the adsorbent can reduce the deterioration rate of renal function while maintaining the normal function of gastro-intestinal tract to avoid dialysis treatment. Preferably, the polyacrylonitrile-based activated carbon fibers have the average diameter of 5-10 μm. Preferably, the polyacrylonitrile-based activated carbon fibers have an average length of more than 20 μm. Preferably, the polyacrylonitrile-based activated carbon fibers have the BET specific surface area in a range of 900 m²/g to 1500 m²/g. Preferably, the polyacrylonitrile-based activated carbon fibers have the total acidic group content in a range of 1.2 meq/g to 1.7 meq/g or the total basic group content in a range of 1 meq/g to 1.7 meq/g; and more preferably, the polyacrylonitrile-based activated carbon fibers have the total acidic group content in a range of 1.253 meq/g to 1.7 meq/g or the total basic group content in a range of 1.26 meq/g to 1.685 meq/g.

The polyacrylonitrile-based activated carbon fibers contained in the adsorbent of the present disclosure can be prepared by oxidizing the polyacrylonitrile-based carbon fiber cloth (Panex® 30, from Zoltek Companies, Inc.); treating the oxidized polyacrylonitrile-based carbon fiber cloth with the carbon dioxide gas containing water vapor at the temperature in a range of 700° C. to 1000° C. for 1 to 60 minutes; and grinding the carbon fiber thus treated. In the present disclosure, there is no specific limit on the polyacrylonitrile-based carbon fiber cloth, that is a carbon fiber cloth which may or may not contain Rayon or petroleum pitch can be used to prepare the carbon fibers of the present disclosure.

The adsorbent of the present disclosure may further comprise a plurality of activated particles such as silver, gold, aluminum, lead, zinc, copper or titanium dioxide particles attached to surfaces of the polyacrylonitrile-based activated carbon fibers, such that the adsorbent may have the ability to reduce the bacteria in intestinal tract and thereby further reducing the concentration of indoxyl sulfate in serum of rat. The activated particles may preferably have a diameter in a range of 1 nm to 500 μm.

It should be understood that the above detailed description and specific examples are given by way of illustration only and are not limitative of the present disclosure. Numerous variations and modifications within the spirit of the present disclosure are intended to be included within the scope of the appended claims.

What is claimed is:

1. An adsorbent for reducing uremic toxins in vivo, comprising:
polyacrylonitrile-based activated carbon fibers having the following properties:
(a) an average diameter ranging from 5 μm to 30 μm;
(b) a BET specific surface area of more than 390 m²/g; and
(c) a total acidic group content of larger than 1.2 meq/g or a total basic group content of larger than 1 meq/g.

2. The adsorbent for reducing uremic toxins in vivo as claimed in claim 1, wherein the polyacrylonitrile-based activated carbon fibers have the total acidic group content ranging from 1.2 meq/g to 1.7 meq/g or the total basic group content ranging from 1 meq/g to 1.7 meq/g.

3. The adsorbent for reducing uremic toxins in vivo as claimed in claim 1, wherein the polyacrylonitrile-based activated carbon fibers have the total acidic group content ranging from 1.253 meq/g to 1.7 meq/g or the total basic group content ranging from 1.26 meq/g to 1.685 meq/g.

4. The adsorbent for reducing uremic toxins in vivo as claimed in claim 1, wherein the polyacrylonitrile-based activated carbon fibers have an average length of more than 20 μm.

5. The adsorbent for reducing uremic toxins in vivo as claimed in claim 1, wherein the polyacrylonitrile-based activated carbon fibers are prepared by treating an oxidized polyacrylonitrile-based carbon fiber material with a carbon dioxide gas containing water vapor at a temperature ranging from 700° C. to 1000° C. for 1 to 60 minutes.

6. The adsorbent for reducing uremic toxins in vivo as claimed in claim 1, wherein the polyacrylonitrile-based activated carbon fibers are prepared by oxidizing a polyacrylonitrile-based carbon fiber cloth which contains 90 wt % of polyacrylonitrile and 10 wt % of Rayon or petroleum pitch, and treating the oxidized polyacrylonitrile-based carbon fiber cloth with carbon dioxide gas containing water vapor at a temperature ranging from 700° C. to 1000° C. for 1 to 60 minutes.

7. The adsorbent for reducing uremic toxins in vivo as claimed in claim 1, wherein the polyacrylonitrile-based activated carbon fibers have the average diameter of 5 μm to 10 μm.

8. The adsorbent for reducing uremic toxins in vivo as claimed in claim 1, wherein the polyacrylonitrile-based activated carbon fibers have the BET specific surface area ranging from 900 m²/g to 1500 m²/g.

9. The adsorbent for reducing uremic toxins in vivo as claimed in claim 1, further comprising a plurality of activated particles attached to surfaces of the polyacrylonitrile-based activated carbon fibers; wherein the plurality of activated particles are selected from the group consisting of silver particles, gold particles, aluminum particles, lead particles, zinc particles, copper particles and titanium dioxide particles.

10. The adsorbent for reducing uremic toxins in vivo as claimed in claim 9, wherein the plurality of activated particles have a diameter ranging from 1 nm to 500 μm.
